(19) United States
(12) Patent Application Publication
(10) Pub. No.: US 2003/0130735 A1
(43) Pub. Date: Jul. 10, 2003

(54) Graft device and methods of use

(76) Inventor: Roger Rogalski, South Lake Tahoe, CA (US)

Correspondence Address: IAN F. BURNS & ASSOCIATES, 1575 DELUCCHI LANE, SUITE 222, RENO, NV, 89502, US

(21) Appl. No.: 10/045,890

(22) Filed: Jan. 9, 2002

(51) Int. Cl. A61F 2/08

(52) U.S. Cl. 623/13.15; 623/13.14

(57) ABSTRACT

The present invention provides a graft device and methods of use. The graft device comprises a protective surface for protecting a tissue portion of a graft from damage by surgical instruments during a surgical operation to attach the graft to a bone. The graft device also comprises a binding surface that is adapted to abut and control the tissue portion of the graft.

### BACKGROUND OF THE INVENTION

1. Field of Invention

This invention relates to medical devices, more specifically to a device and method for binding grafts.

2. Description of Related Art

Anterior cruciate ligament (“ACL”) reconstruction is a common surgical procedure that intends to restore knee stability following an ACL ligament tear. This common reconstructive surgical procedure substitutes a deficient tissue with a healthy tissue in a form of a graft from another patient or from another body part of the same patient. The type of graft is well known and could be, among many other types, autologous ligament grafts, allografts, or artificial grafts. One type of graft used in ACL reconstruction is a bone-tendon-bone (BTB) ligament graft, which is harvested from the patella and tibia. A BTB ligament graft comprises a tissue portion and bone blocks at each end of the graft. The reconstructive procedure usually includes anchoring a graft in a pre-drilled bone tunnel with an interference fit screw. The surgical technique can either be open or arthroscopic of a one incision or two-incision fashion. The endoscopic (one incision technique) is rapidly gaining popularity. One of the pitfalls with the one incision endoscopic technique is poor visual access during an insertion of the interference fit screw in a femoral tunnel. This poor visual access is caused by an obstruction from a tissue portion of the graft and it can cause eccentric screw placement or damage to the tissue portion of the graft by the sharp threads of the screw. One possible result is amputation of the tissue portion of the graft from the bone block.

Inventors have attempted to reduce the danger of graft damage during surgery by altering the design of the interference fit screw. U.S. Pat. No. 5,496,326 (Johnson) discloses an interference screw with a rounded tip, shallower thread depth, and thread that has a rounded exterior edge. U.S. Pat. No. 5,984,966 (Kiema et al.) discloses an implant manufactured of a bioabsorbable material, the implant being pushed into a hole or drill canal made into a bone to fix a bone graft into the drill hole. However, with these inventions, the tissue portion of the graft remains subjected to the risk of coming into physical contact with the interference screw, as no barrier exists between the graft and the interference screw.

Other inventors have resorted to encasing the interference screw to minimize the danger the interference screw poses to the graft. U.S. Pat. No. 5,658,289 (Boucher et al.) discloses a graft protection device having an elongated, cannulated driver for advancing a cannulated device along a guiding wire during a surgical operation. U.S. Pat. No. 5,904,685 (Walawalkar) discloses an apparatus for fixation of a graft by screw insertion having a cannulated sheath for guiding a screw into a tunnel in a surgical site. However, these devices fail to protect the graft from cuts and nicks caused by contact with the devices when the devices are introduced into the surgical site. Additionally, the graft is exposed to the same danger when the device is pulled out of the surgery site after the installation of the screw. Furthermore, these devices are expensive to manufacture, bulky, and require surgeons, who are accustomed to performing reconstructive surgery the traditional way, to modify their technique to accommodate the use of a bulky device when installing the interference screw.

Other inventors have approached the problem by eliminating the use of interference screws altogether. U.S. Pat. No. 5,645,588 (Graf et al.) discloses an attachment device adapted to secure the graft to the bone, the attachment device having an elongated body and at least one element for attaching the graft or a graft connection element to the body. However, this device makes reconstructive surgery more difficult and more time-consuming. This device also requires surgeons, who are accustomed to performing reconstructive surgery the traditional way, to modify their technique. These devices do not allow for a strong attachment of the graft to the bone as previously provided by the interference screw.

Other prior art related to the field of the present invention pertains to improving the fixation between the ligament and the bone. U.S. Pat. No. 6,214,007 (Anderson) discloses a device having a collar with side wall openings and a screw with conical head and blunt threads. The device is designed to firmly hold tissue graft to the bone by allowing the screw threads and collar to compress tissue portion of the graft into the sidewalls of the bone tunnel and capturing graft tails using the interface of the screw head and collar. U.S. Pat. No. 6,264,694 (Weiler) discloses a fixation device with a spherical member having a thorough bore which enables it to be tied to the end of a soft tissue graft thereby enabling the graft to be pulled into the bone tunnel and be secured within the bone tunnel by an interference screw. These prior art do not address the problem regarding the graft coming into contact with the interference screw during the surgery. These inventions also do not aid in easing the insertion of the interference screw during the surgery as they allow the graft to obstruct the path of the interference screw.

In summary, there is a long felt but unmet need for improving the poor visual access caused by graft obstruction during the insertion of the interference screw in the femoral tunnel during a reconstructive surgery. There has also been a long felt and unmet need for preventing eccentric interference screw placement and damage to tissue portion of graft from the interference screw. Prior art has unsuccessfully attempted to fulfill these needs by altering the design of the interference screw, encasing the interference screw, eliminating the interference screw altogether, and providing devices that modify the traditional reconstructive surgery technique. The present invention provides a device and method that fulfills these needs by approaching the problem in a way that is not suggested by the prior art.

### SUMMARY OF INVENTION

Advantages of the Invention

An advantage of the present invention is that it provides a barrier that prevents tissue portions of grafts from coming into contact with an interference screw, which can cut or damage the tissue portion of grafts.

Another advantage of the present invention is that it enhances visual access to the path of the interference screw during a reconstructive surgery.

Another advantage of the present invention is that it allows for a substantially exact screw placement in the bone tunnel during reconstructive surgery.

Another advantage of the present invention is that it provides a graft device with a guide mark that guides surgeons as to the direction of the interference screw when it is inserted into a bone tunnel.

Another advantage of the present invention is that it provides a graft device that is easy and inexpensive to manufacture.

Another advantage of the present invention is that it provides a graft device that is simple to use and does not require surgeons, who are accustomed to performing reconstructive surgery the traditional way, to modify their technique.

Another advantage of the present invention is that it provides a graft device that can be quickly installed on tissue portion of graft thereby allowing the operating surgeon to work on different task.

Another advantage of the present invention is that it provides a graft device that makes reconstructive surgery safer, easier, and less time consuming.

Another advantage of the present invention is that it provides a graft device that may be used with an interference screw, which provides strong attachment between a graft and a bone.

Another advantage of the present invention is that it provides a graft device that produces little or no negative side effects. These and other advantages of the present invention may be realized by reference to the remaining portions of the specification, claims, and abstract.

### BRIEF DESCRIPTIONS OF THE INVENTION

The present invention comprises a graft device and methods of use. The graft device comprises a protective surface for protecting a tissue portion of a graft from damage by surgical instruments during a surgical operation to attach the graft to a bone. The graft device also comprises a binding surface that is adapted to abut the tissue portion of the graft.

The above description sets forth, rather broadly, the more important features of the present invention so that the detailed description of the preferred embodiment that follows may be better understood and contributions of the present invention to the art may be better appreciated. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description or as illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1 is substantially a perspective view of an ACL reconstruction.

FIG. 2 is substantially an elevated side view of one embodiment of the graft device of the present invention.

FIG. 3 is substantially a perspective view of an ACL reconstruction further showing one embodiment of the graft device of the present invention.

FIG. 4 is substantially a cross-sectional view of one embodiment of the graft device of the present invention attached to a tissue portion of a graft.

FIG. 5 is substantially a side view of another embodiment of the graft device of the present invention.

FIG. 6 is substantially a perspective view of another embodiment of the graft device of the present invention.

FIG. 7 is substantially a perspective view of the embodiment of the present invention shown in FIG. 6 with the device in an open position.

FIG. 8 is substantially a perspective view of the embodiment of the present invention shown in FIGS. 6 and 7 attached to a tissue portion of a graft.

FIG. 9 is substantially a perspective view of yet another embodiment of the graft device of the present invention attached to a tissue portion of a graft.

FIG. 10 is substantially a perspective view of the embodiment of the present invention shown in FIG. 9.

FIG. 11 is substantially a perspective view of an additional embodiment of the graft device of the present invention.

FIG. 12 is substantially a perspective view of the embodiment of the graft device of the present invention shown in FIG. 11.

FIG. 13 is substantially a perspective view of the embodiment of the present invention shown in FIG. 11 attached to a tissue portion of a graft.

FIG. 14 is substantially a perspective view of an additional embodiment of the graft device of the present invention.

FIG. 15 is substantially a perspective view of the embodiment of the present invention shown in FIG. 14 attached to a tissue portion of a graft.

FIG. 16 is substantially a perspective view of an additional embodiment of the graft device of the present invention.

### DESCRIPTION OF THE PREFERRED EMBODIMENT

In the following detailed description of the preferred embodiments, reference is made to the accompanying drawings, which form a part of this application. The drawings show, by way of illustration, specific embodiments in which the invention may be practiced. It is to be understood that other embodiments may be utilized and structural changes may be made without departing from the scope of the present invention.

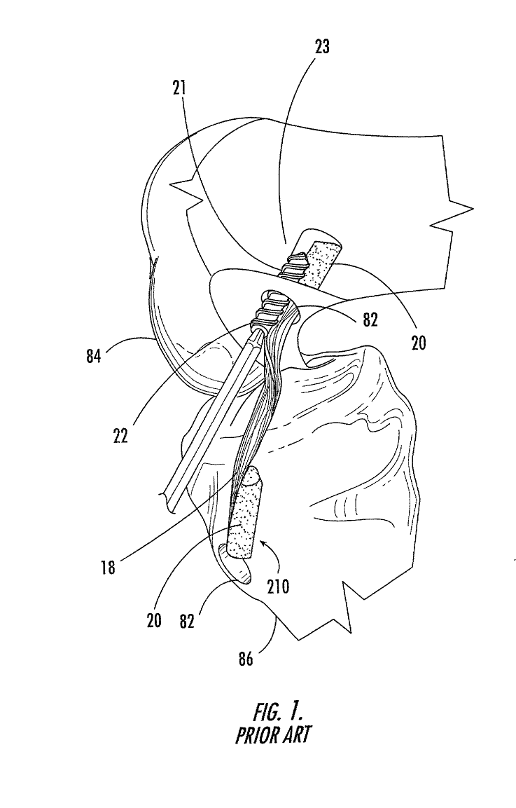

Referring to FIG. 1, the present invention is used in reconstructive surgical operations, such as reconstruction of anterior cruciate ligaments (ACL) and posterior cruciate ligaments (PCL). Generally, ACL and PCL procedures involve preparing a bone tunnel 82 through the tibia 86 and adjacent femur 84, placing a graft 210 that extends between the two bone tunnels 82, and securing each end of graft 210 within its respective tunnel 82. One type of graft used in ACL reconstruction is a bone-tendon-bone (BTB) ligament graft, which is harvested from the patella and tibia. Other types of grafts include autologous ligament grafts, allografts, artificial grafts, and soft tissue grafts such as semitendinosus, hamstring, achilles, or quadriceps. A BTB ligament graft comprises a tissue portion 18 and bone blocks 20 at each end of graft 210. Bone blocks 20 are fixed within its respective tunnel 82 by interference screws 22 secured within each tunnel 82 between tunnel wall 23 and bone block 20. Interference screw 22 is aligned parallel to the axis of tunnel 82 and holds bone bock 20 in tunnel 82 by holding it against tunnel wall 23 opposite the screw and by engaging the bone block 20 and adjacent tunnel wall 23 with the screw threads 21.

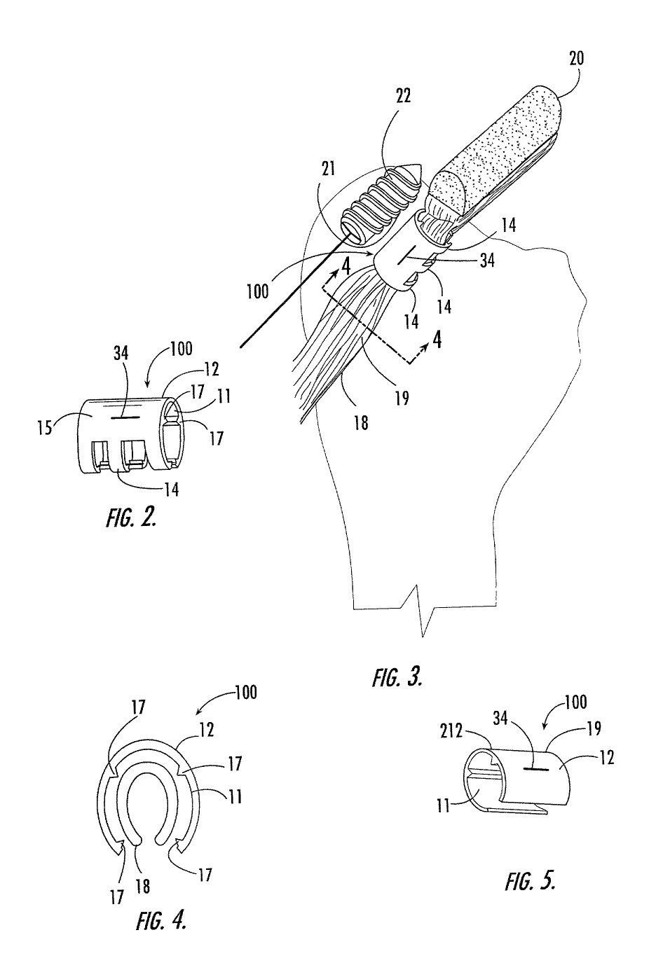

Turning now to FIGS. 2 and 3, the present invention comprises a graft device, generally indicated by reference number 100. The graft device 100 comprises a protective surface 12 and a tissue surface 11. The protective surface 12 provides a barrier that protects a tissue portion of a graft 18 from damage by surgical instruments, such as threads 21 of screw 22, during a reconstructive surgical operation. Preferably, protective surface 12 is resistant to penetration by surgical instruments used in surgical operation. Tissue surface 11 abuts tissue portion of graft 18 and binds graft fibers 19 together. Tissue portion of graft 18 may be comprised of hundreds of individual fibers or strands 19. By binding fibers 19 together, the present invention prevents the fibers from spreading out and interfering with the surgical procedure. Not only are the fibers less likely to be damaged by surgical instruments because they are held in one place, the strands also do not obstruct the surgeons view of bone tunnel 82. This allows surgeons to more quickly and accurately place screw 22 in tunnel 82.

Graft device 100 further comprises an attachment structure 14 that attaches graft device 100 to tissue portion of graft 18. In the embodiment shown in FIGS. 2 and 3, graft device 100 comprises a central portion 15 and a plurality of fingers 14 attached to central portions. Fingers 14 hold tissue portion of graft 18 within central portion 15. In the preferred embodiment, three fingers 14 are provided on each side of graft device 100. Graft 18 is held between central portion 15 and fingers 14 when graft device 100 is attached to tissue portion of graft 18.

As seen in FIG. 4, tissue portion of graft 18 may be partially compressed by fingers 17 when graft device 100 is attached to the tissue. Graft device 100 may be folded along its lateral axis so that it has a U-shaped cross section. This helps graft device 100 maintain a clamping pressure on tissue portion of graft 18.

In the preferred embodiment, the graft device 100 is made of bioabsorbable material, such as polyglycolide (PGA) copolymers, poly-(D,L-lactide-co-glycolide) (PDLLA-co-PGA), poly(D,L-lactide) (PDLLA), or stereocopolymers of Poly-(L-lactide) and poly-(D,L-lactide) (PLLAco-PDLLA) with varying ratios of the L and D, L parts. Other bioabsorbable materials that are well known in the art may also be used. Examples of additional materials that may be used with the present invention are disclosed in article titled “Biodegradable Implants in Sports Medicine: The Biological Base” by Weiler et al. (Arthroscopy: The Journal of Arthroscopic and Related Surgery, Vol 16, No 3 (April), 2000: pages 305-321). The bioabsorbable material is preferably quickly absorbed by a patient after the surgical procedure, provides sufficient structural rigidity to bind and protect tissue portion of graft 18, and can be repeatedly bent and straightened without fracturing.

As seen in FIGS. 2, 3, 5, 6, 8, 11-13, graft device 100 further comprises a guide mark 34 for assisting in the placement of interference screw 22. As surgeon positions the interference screw 22, guide mark 34 provides a visual reference by which the position and alignment of screw 22 can be judged. This helps the surgeon to position screw 22 so that it is parallel to the axis of tunnel 82. Guide mark 34 could either be engraved on graft device 100 or implanted on graft device 100 using a bioabsorbable material described above.

As seen in FIGS. 2, 4-7, and 11-12, graft device 100 further comprises of at least one projection 17 attached to the tissue surface 11. Projection 17 may have a large number of different configurations, such as spike-shaped or ridge-shaped. When graft device 100 is attached to a tissue portion of graft 18, projection 17 helps hold tissue portion of graft 18 within central cavity 15 and prevents the graft device from rotating on tissue portion of grafts. The number of projections 17 may vary.

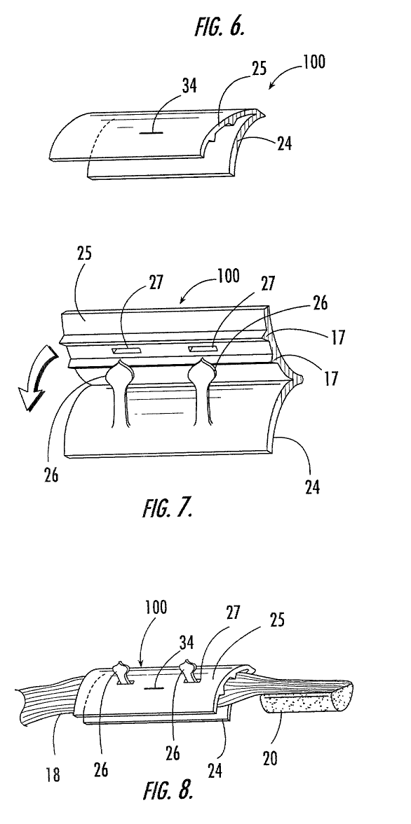

FIG. 5 shows another embodiment wherein the graft device 100 comprises a sleeve 212 having a substantially C-shaped cross section. C-shaped cross section defines a central cavity, which is configured to receive and hold tissue portion of graft 18. Sleeve 212 also comprises protective surface 12 and tissue surface 11.

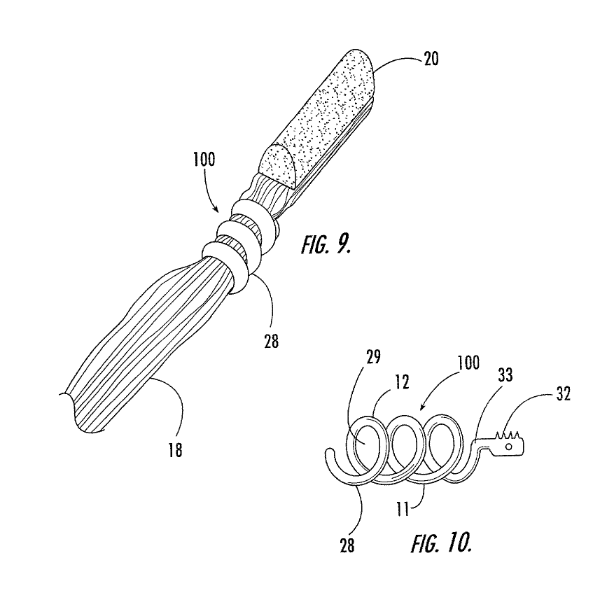

FIG. 6 illustrates an alternative embodiment of graft device 100. In this embodiment, graft device 100 comprises a first flap 24 and a second flap 25. First flap 24 is foldably attached to second flap 25 along a longitudinal axis of graft device 100. In normal operation, graft 18 is placed between first flap 24 and second flap 25 so that tissue portion of grafts is substantially enclosed within graft device 100. In the preferred embodiment, seen in FIGS. 7 and 8, graft device 100 further comprises at least one tab 26 attached to first flap 24. The second flap 25 has at least one hole 27 to allow tab 26 to extend through hole 27. When graft device 100 is attached to tissue portion of graft 18, tab 26 extends through tissue portion of graft 18 and hole 27. Since tissue portion of graft 18 comprises a number of individual strands or fibers, tab 26 can be inserted through tissue portion of graft 18 without causing significant damage to the tissue portion of graft. Tab 26 may then be bent over to further secure flaps 24 and 25 to tissue portion of draft 18. Tab 26 may comprise a barb or other similarly shaped structure that prevents tab 26 from inadvertently disengaging from hole 27. Tab 26 may be integrally made from the flap or can be a separate structure from flap 24 such as a pin with clover-shaped head. Flaps 24 and 25 are preferably rectangular and have an arc-shaped cross section along its lateral axis. This shape provides greater structural rigidity and secures tissue portion of graft 18 away from surgical instruments. Of course, number of tabs 26 and holes 27 may vary.

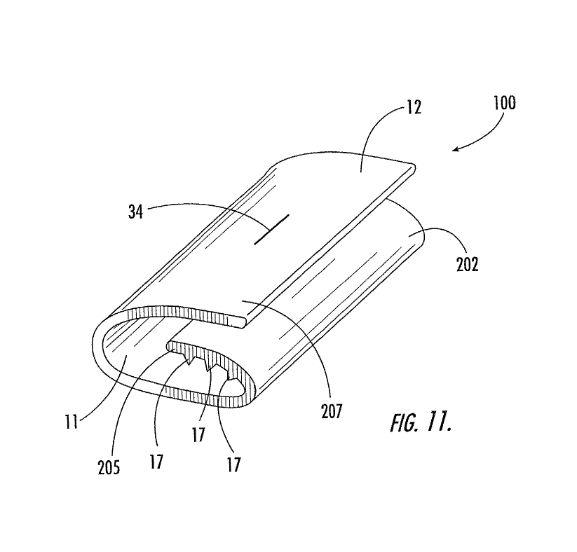

Referring now to FIG. 9, as another alternative embodiment, graft device 100 comprises a helix 28 formed from a number of rotations of flexible bioabsorbable material. Helix 28 forms a central cavity 29. In normal operation, tissue portion of graft 18 is enclosed within central cavity 29 by winding helix 28 around tissue portion of graft 18. Helix 28 comprises protective surface 12 on surface of helix distant from tissue portion 18 and tissue surface 11 on helix surface adjacent to tissue portion 18. The number of helical coils may vary. Alternatively, helix 28 can be a pre-molded bioabsorbable material and can be attached to tissue portion of graft 18 by sliding it over an end 210 of graft.

As seen in FIG. 10, helix 28 may comprise a projection 33. Projection 33 prevents draft device 100 from moving relative to tissue portion of graft 18. Projection 33 may be positioned on end 32 of helix 28. End 32 may be substantially parallel to the longitudinal axis of graft 210. In this embodiment, projection 33 binds to tissue portion of graft 18, thereby and inhibiting motion of graft device 100. An alternative embodiment does not include a projection. In this alternative embodiment, tension secures graft device 100 to tissue portion of graft 18.

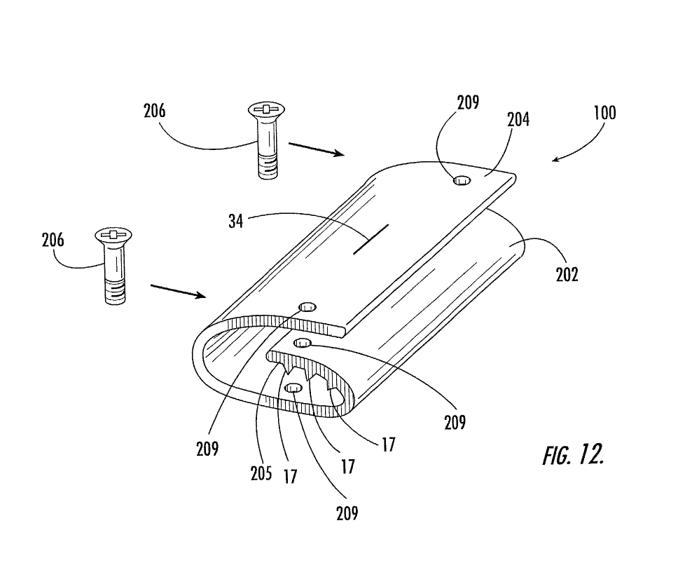

FIG. 11 shows yet another embodiment of graft device 100. In this embodiment, graft device 100 comprises a flexible material 202 with a first end 205 and a second end 207. To attach this embodiment of graft device 100 to tissue portion of graft 18, flexible material 202 is wrapped around tissue portion of graft so that first end 205 is in close relative proximity to second end 207, thereby forming a cylindrical tube-like structure.

In one embodiment, flexible material 202 is rigid enough to hold tissue portion of graft 18 inside of the flexible material 202 without attaching first end 205 to second end 207. In an alternative embodiment, ends 205 and 207 are attached by using mechanical fasteners 206.

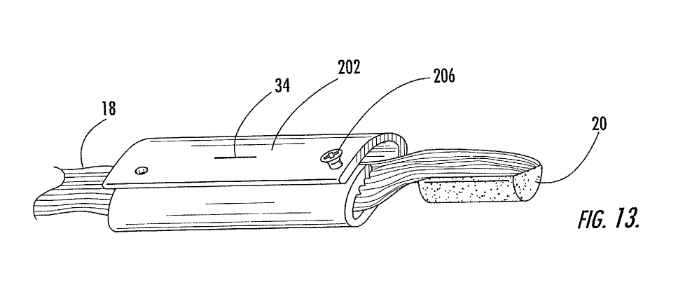

Referring now to FIGS. 12 and 13, flexible material 202 may comprise holes 209 and fasteners 206. In normal operation, once flexible material 202 has been wrapped around tissue portion of graft 18, fasteners 206 are inserted into holes 209 and first and second ends 205 and 207 are held together. Type of fastener 206 may be any fastener well known in the art. In the preferred embodiment, fasteners 206 are long enough to penetrate through flexible material 202 and tissue portion of graft 18.

Fasteners 206 preferably have a broad flat head to hold the fastener onto the flexible material 202 and prevent the fastener 206 from sliding through flexible material 202. Fasteners 206 are preferably made of bioabsorbable material discussed above. The number of fasteners 206, holes, their positions, and structures may be varied and still achieve the advantages of the present invention.

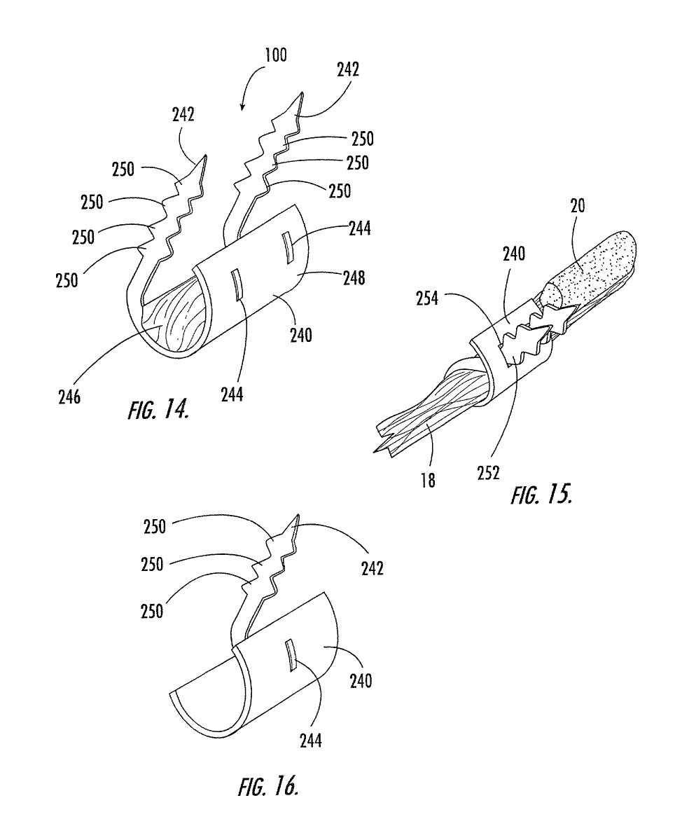

FIGS. 14, 15, and 16 show another embodiment of the graft device 100. In this embodiment, graft device 100 comprises a flexible material 240 and at least one strap 242. Flexible material 242 includes a binding surface 246 and a protective surface 248. Binding surface 246 is preferably concave. Binding surface 246 is adapted to surround tissue portion of a graft. Protective surface 248 is adapted to protect tissue portion 18 of graft from surgical instruments during insertion of graft to a bone. Strap 242 comprise of ridges 250. Number of ridges 250 may vary. In one embodiment shown in FIG. 14, two straps 242 are attached to flexible material 240. In another embodiment shown in FIG. 16, one strap 242 is attached to flexible material 240. Of course, the number of straps may vary. Strap 242 and flexible material 240 may be molded together. Thus, strap 242 may be integrally attached to flexible material 240. Strap 242 may also be attached to flexible material 240 in a manner well known in the art such as using an adhesive, welding, or soldering.

Flexible material 240 may comprise of at least one hole 244 to accommodate at least one strap 242. When graft device is attached to tissue portion 18 of a graft, tissue portion 18 is positioned on binding surface 246 of flexible material 242. Strap 242 is inserted into corresponding hole 244. Strap 242 is pulled until tissue portion 18 is substantially surrounded by flexible material 240. Strap 242 may be adjusted preferably providing snug fit of tissue portion 18 within flexible material 240. Ridges 250 prevent strap 242 from moving away from hole 244. In the preferred embodiment, excess strap 254 protruding past locking ridge 252 is trimmed.

### CONCLUSION

Although the description above contains many specifications, these should not be construed as limiting the scope of the invention but as merely providing illustrations of some of presently preferred embodiments of this invention. Thus, the scope of the invention should be determined by the appended claims and their legal equivalents rather than by the examples given.

What is claimed is:

1. A graft device, the graft device comprising:
(A) a first flap; and
(B) a second flap, the second flap being foldably attached to the first flap, wherein a tissue portion of a graft is held between the first and second flaps when the graft device is attached to the tissue portion of the graft.

2. The graft device of claim 1, further comprising at least one tab attached to the first flap and a hole in the second flap, wherein the tab extends through hole and holds the first and second flap in a folded position when the graft device is attached to the tissue portion of the graft.

3. A graft device, the graft device comprising a helix, the helix forming a central cavity, the central cavity being configured to receive a tissue portion of a graft, wherein the tissue portion of the graft is enclosed within the helix when the graft device is attached to the tissue portion of the graft.

4. A graft device according to claim 3, further comprising at least one projection, the projection being in contact with the tissue portion of the graft when the graft device is attached to the tissue portion of the graft.

5. A graft device, the graft device comprising flexible material, the flexible material having a first and second end, the flexible material being configured to be wrapped around a tissue portion of a graft, wherein the tissue portion of the graft is substantially enclosed within the flexible material when the graft device is attached to the tissue portion of the graft.

6. The graft device of claim 5, wherein the first end overlaps the second end, and further comprising at least one fastener adapted to attach the first end to the second end.

7. The graft device according to claim 5, further comprising at least one projection attached to the flexible material, wherein the projection is in contact with the tissue portion of the graft when the flexible material is attached to the tissue portion of the graft.

8. The graft device of claim 5, further comprising at least one strap attached to the flexible material and at least one hole on the flexible material, the strap being adapted to extend through the hole to secure the flexible material on the tissue portion of the graft.

9. The graft device of claim 8, wherein the strap comprises at least one ridge, the ridge being adapted to secure the strap into the hole.

10. A method for protecting a graft during a surgical procedure to attach the graft to a bone, the method comprising:
(A) providing a graft;
(B) providing a graft device, the graft device comprising a tissue surface on the interior of the graft device, and a protective surface on the exterior of the graft device; and
(C) attaching the graft device to a tissue portion of the graft.

11. The method of claim 10, further comprising
(A) providing a guide mark; and
(B) moving an interference screw toward a bone tunnel in direction parallel to axis of the guide mark.

12. A graft device, the graft device comprising:
(A) an interior tissue surface, the tissue surface being configured to abut a tissue portion of a graft and bind the tissue portion of graft; and
(B) an exterior protective surface, the protective surface being configured to serve as barrier for the tissue portion of the graft thereby protecting the tissue portion of the graft from damage by surgical instruments during a surgical operation to attach the graft to a bone.

13. The graft device according to claim 12 further comprising an attachment structure, the attachment structure being configured to attach the graft device to the tissue portion of the graft.

14. The graft device according to claim 12, further comprising a guide mark affixed on the protective surface, the guide mark being configured to provide a visual reference to assist insertion of an interference screw in a bone tunnel.

15. The graft device of claim 13, wherein the attachment structure comprises a central portion and at least one finger attached to the central portion, the finger extending from the central portion so as to define a cavity between the finger and the central portion, wherein the tissue portion of the graft is held between the central portion and the finger when the graft device is attached to the tissue portion of the graft.

16. The graft device of claim 13, wherein the attachment structure comprises an inner surface having a substantially C-shaped cross section defining a cavity, wherein the tissue portion of the graft is held inside the cavity when the graft device is attached to the tissue portion of the graft.

17. The graft device of claim 12, further comprising a helix, wherein the tissue portion of the graft is enclosed within the helix when the graft device is attached to the tissue portion of the graft.

18. The graft device of claim 17, wherein at least one end of the helix is substantially straight and further comprising at least one tooth perpendicularly attached on the helical end, the tooth being pointed and configured to grasp onto the tissue portion of the graft thereby securing the attachment of the helix to the tissue portion of the graft.

19. A graft device according to claim 12, further comprising a bioabsorbable material.

20. A graft device according to claim 13, wherein the attachment structure comprises at least one tab attached to the tissue surface, the tab being displaced at least partially perpendicular to the tissue surface.

21. A graft device, the graft device comprising a sleeve, the sleeve having a substantially C-shaped cross section defining a cavity, the cavity being configured to receive the issue portion of a graft and hold the tissue portion of the graft when the graft device is attached to the tissue portion of the graft.

22. The graft device of claim 21, further comprising a central portion and at least one finger integrally attached to the central portion.

23. The graft device of claim 21, further comprising fingers, the fingers extending from the sleeve, the fingers being integrally attached to the sleeve, the fingers comprising a tab at an end.

24. A method for reconstructing an anterior cruciate ligament comprising:
(A) harvesting a graft having a bone block at both ends of the graft;
(B) drilling a femoral tunnel;
(C) drilling a tibial tunnel;
(D) providing a graft device comprising a tissue surface on the interior of the graft device, a protective surface on the exterior of the graft device, and an attachment structure;
(E) attaching the graft device onto a tissue portion of the graft;
(F) positioning one end of the graft into the femoral tunnel; and
(G) binding the bone block of the graft onto a tunnel wall using an interference screw.

25. The method of claim 24, further comprising
(A) providing a guide mark; and
(B) introducing an interference screw toward the bone tunnel in direction parallel to axis of the guide mark.
